(19) United States
(12) Patent Application Publication
(10) Pub. No.: US 2002/0086294 A1
(43) Pub. Date: Jul. 4, 2002

(54) Device and method for tracking conditions in an assay

(76) Inventors: Richard N. Ellson, Palo Alto, CA (US); Mitchell W. Mutz, Palo Alto, CA (US); David L. Harris, Mountain View, CA (US)

Correspondence Address: REED & ASSOCIATES, 800 MENLO AVENUE, SUITE 210, MENLO PARK, CA, 94025, US

(21) Appl. No.: 09/751,231

(22) Filed: Dec. 29, 2000

(51) Int. Cl. C12Q 1/68; G01N 33/537; C12M 1/34

(52) U.S. Cl. 435/6; 435/7.92; 435/287.2

(57) ABSTRACT

The invention provides a device comprising a substrate having a plurality of different molecular probes attached to a surface thereof and an integrated indicator that exhibits a response when exposed to a condition to which the substrate may be exposed. Each different molecular probe is selected to interact with a different corresponding target, and the indicator response is detectable after removing the indicator from the condition. Alternatively, a substrate is provided having a plurality of molecular probes attached to a surface thereof and a plurality of different integrated indicators. Each indicator is selected to exhibit a response when exposed to one of a plurality of conditions to which the substrate may be exposed. The inventive devices are typically used for biomolecular, or more specifically, nucleotidic assays. The invention also provides for various apparatuses and methods for assaying a sample using the inventive devices.

### TECHNICAL FIELD

This invention relates generally to assay condition tracking. More particularly, the invention relates to devices comprising a substrate having surface-attached molecular probes to carry out an assay and having integrated indicators responsive to environmental conditions associated with the assay.

### BACKGROUND

The formation of high-density biomolecular arrays, e.g., oligonucleotidic or polynucleotidic arrays, is well known in the art. For example, U.S. Pat. No. 5,744,305 to Fodor et al. describes arrays of oligonucleotides and polynucleotides. The arrays are described as consisting of a plurality of different oligonucleotides attached to a surface of a planar non-porous solid support at a density exceeding 400 different oligonucleotides/cm². This patent discloses that photolithographic techniques associated with semiconductor processing may be employed in order to form arrays of such high density. In addition, focused acoustic energy can be used to form such arrays as described in detail in patent application U.S. Ser. No. 09/669,996 (“Acoustic Ejection of Fluids From a Plurality of Reservoirs”), inventors Ellson, Foote and Mutz, filed on Sep. 25, 2000 and assigned to Picoliter, Inc. (Cupertino, Calif.).

High-throughput assays, such as oligonucleotide hybridization experiments, often take place on an array of test sites on an assay substrate. Assay substrates may, for example take the form of glass plates, microscope slides or microtiter well plates, and test sites may be formed as features on such substrate surfaces. Many arrays are formed having large feature-to-feature and array-to-array variations, and such variations adversely affect the reproducibility of experimental conditions and results. Consequently, the variation in the assay substrate increases the difficulty in comparing results from experiment to experiment, in effect increasing the noise-to-signal ratio in these experiments.

In order to ensure the accuracy of these experiments, a control sample is usually used in conjunction with a test sample. The control sample may be used to determine the degree of feature-to-feature and array-to-array variation. In other words, conducting the experiment with the control sample serves to calibrate the assay results. This is disadvantageous for a number of reasons, one of which being that the control sample itself may be a source of variability. That is, if there is excessive variation in the control sample, the control sample is no longer useful as a calibration tool. Moreover, single feature controls typically indicate merely whether a hybridization event has occurred. If no hybridization event occurs, such controls do not provide additional information to assess why no hybridization occurs or guide the user directly to a more successful experiment.

For example, one widely used method for managing variability in arrays involves applying two samples, a control and a test sample, simultaneously to the same array. By labeling each sample with a different tag, such as different colors of fluorescent markers, the amount of binding of each tag can be measured independently at each site. Such labeling with different markers has been described, e.g., in U.S. Pat. No. 5,770,358 to Dower et al., U.S. Pat. No. 5,800,992 to Fodor et al. and U.S. Pat. No. 5,830,645 to Pinkel et al. By comparing the signal of the test-sample interaction with a test site and the signal of the control-sample interaction with the same test site, a source of variability is eliminated. However, using different tags on the test and control samples introduces a new source of variability. The relative chemical activity of the test and control samples may be altered, which in turn changes the reaction rate of the two samples with the test site. As a result, another experiment may be required to determine the effect of using different tags. This can be carried out by repeating the experiment using the control and the test samples labeled with switched tags. However, repeating the experiment may reintroduce array-to-array and feature-to-feature variability. Thus, it becomes extremely important to ensure that the repeated experiment is conducted in a substantially identical manner to the first experiment.

The above example illustrates the need to address feature-to-feature and array-to-array variability and the need to ensure the assay is performed under uniform conditions. However, it is important to note that optimal assay analysis should effectively decouple the array-to-array and feature-to-feature variations and the conditions under which the assay was performed with the array. As the array-forming technology becomes more effective in generating reproducible arrays, the contribution to variation from the assay procedure grows in importance. Thus, in order to ensure that an experiment is repeated with accuracy, it is important to have an accurate record of the previous experimental conditions, irrespective of array-to-array or feature-to-feature variations. In addition, it may be helpful to have an accurate record of the conditions in which arrays are formed.

There are a number of patents that describe integrated devices containing both surface-bound chemical moieties and related information. See, e.g., U.S. Pat. No. 6,030,581 to Virtanen, U.S. Pat. No. 5,872,214 to Nova et al. and U.S. Pat. No. 5,935,786 to Reber et al. In addition, U.S. Ser. No. ______, (“Integrated Device with Surface-Attached Molecular Moieties and Related Machine-Readable Information”), inventors Ellson, Foote and Mutz, filed on Nov. 13, 2000 and assigned to Picoliter, Inc. (Cupertino, Calif.), e.g., describes substrates having a surface adapted for attachment with a plurality of molecular moieties and containing related machine-readable information that facilitates formation and/or use of those moieties, e.g., arrays. While information relating to assay conditions may be contained in these devices, assay conditions must be separately monitored and then converted into information in the devices. This poses a problem particularly where it is desirable to perform assays with different equipment, at different locations or at widely separated times.

Thus, there is a need in the art for improved devices comprising a substrate having a plurality of surface-attached moieties and an integrated indicator that exhibits a response to a condition wherein the response is detectable after removing the indicator form the condition, thereby providing a record of the condition. The condition may relate to the execution of an assay or to the formation of a device to carry out an assay.

### SUMMARY OF THE INVENTION

Accordingly, it is an object of the present invention to provide devices and methods that overcome the above-mentioned disadvantages of the prior art. In one embodiment, the invention provides a device comprising a substrate having a plurality of different molecular probes attached to a surface thereof and an integrated indicator that exhibits a response when exposed to a condition to which the substrate may be exposed. Each different molecular probe is selected to interact with a corresponding target. The probes preferably interact with different targets but in some cases may interact with the same target with differing degrees of affinity. The indicator response is detectable after removing the indicator from the condition. The indicator response to the condition is typically detectable for at least 1 minute after removing the indicator from the condition and is preferably substantially permanently detectable.

In another embodiment, the invention provides a device comprising a substrate having a plurality of molecular probes attached to a surface thereof and a plurality of different integrated indicators. Each indicator is selected to exhibit a response when exposed to one of a plurality of conditions to which the substrate may be exposed. The molecular probes are selected to interact with corresponding targets. Again, the indicator response is detectable after removing the indicator from the condition. In some cases, the molecular probes are selected to interact with corresponding targets when exposed to at least one of the plurality of conditions. In other cases, molecular probes are selected to interact with corresponding targets when exposed to all of the conditions. Furthermore, molecular probes may be selected to interact with corresponding targets when exposed to all of the conditions simultaneously.

The inventive devices are typically used for biomolecular assays. Thus, the probes are typically biomolecular. More specifically, the probes are ordinarily nucleotidic or peptidic. Often, the probes are arranged in a high-density array on the substrate surface, and such an array may comprise at least about 1,000,000 probes per square centimeters of substrate surface. The probes may interact with targets of various types. For example, the targets may represent portions of a single molecule or portions of a single cell. In the case where the probes are nucleotidic, it is preferred that the integrated indicator also comprises a nucleotidic material.

Thus, in still another embodiment, the invention provides a device comprising a substrate having a plurality of nucleotidic molecular probes attached to a surface thereof and an integrated indicator that exhibits a response when exposed to a condition to which the substrate may be exposed. In this embodiment, the nucleotidic molecular probes are selected to interact with corresponding targets. As in the case of the above embodiments, the indicator response in this embodiment is also detectable after removing the indicator from the condition. Preferably, the condition represents a hybridization condition between the probes and targets.

For any of the above-described embodiments, the indicator may exhibit a response to various conditions such as an environmental condition that allows for or prohibits target-probe interaction. These conditions include, for example, temperature, chemical content and chemical concentration. Although the indicator response may be magnetic and/or electrically detectable, the response is preferably optically detectable and optimally fluorescently detectable.

The invention also provides for various apparatuses and methods for assaying a sample using the inventive devices as described above. The assay is carried out by first exposing any of the above described devices to an assay condition by contacting a sample with the probes attached to the substrate surface of the device. Depending on whether the indicator exhibits a response, the assay further involves detecting for probe-target interactions. That is, the presence or the absence of an indicator response serves as a quality control measure for the assay.

In a further embodiment, the invention provides a device comprising a substrate having a surface adapted for attachment to a plurality of molecular moieties and an integrated indicator that exhibits a response when exposed to a condition. As is the case with the above-described embodiments, the indicator response is detectable after removing the indicator from the condition. This embodiment may serve as a precursor to the above-described embodiment. Accordingly, the indicator for this embodiment may exhibit a response to a condition that is or is not suitable for attaching the plurality of molecular moieties to the substrate surface. Also provided are an apparatus and method for attaching molecular moieties to the substrate.

### BRIEF DESCRIPTION OF THE DRAWINGS

FIGS. 1A, 1B and 1C, collectively referred to as FIG. 1, schematically illustrate a device of the present invention comprising a substrate in the form of a single disk having molecular probes and an integrated indicator attached to a top surface of the disk.

FIG. 1A shows the top view of the disk.

FIG. 1B illustrates a cross-sectional view of the device along dotted line A.

FIG. 1C illustrate a bottom view of the disk.

FIGS. 2A, 2B, 2C, and 2D, collectively referred to as FIG. 2, illustrate in schematic view another version of the inventive device wherein the substrate comprises a cartridge containing a magnetic disk and having an exterior surface formed by a well plate having an array of integrated indicators thereon and molecular moieties attached to an interior surface of the wells of the well plate.

FIG. 2A shows a top view of the cartridge.

FIG. 2B illustrates in cross-sectional view of the cartridge of FIG. 2A along dotted line B.

FIG. 2C illustrates the cross sectional view of the cartridge of FIG. 2A along dotted line C.

FIG. 2D illustrates the bottom view of the cartridge.

FIGS. 3A, 3B, 3C, collectively referred to as FIG. 3, schematically illustrate in simplified cross-sectional view another version of the inventive device in the form of a slide having two opposing surfaces.

FIG. 3A shows the top view of the slide having probes and integrated indicators attached thereto, and

FIG. 3B illustrates a cross-sectional view of the slide of FIG. 3A along dotted line E.

FIG. 3C shows the bottom view of the slide having an optional memory chip.

FIG. 4A, 4B, 4C and 4D, collectively referred to as FIG. 4, schematically illustrate in simplified cross sectional view a method for carrying out an assay using the inventive device.

In FIG. 4A, a device is shown having a construction similar to that illustrated in FIG. 3.

FIG. 4B illustrates the loading of the device into a hybridization chamber wherein a fluid sample comes into contact with the probes.

FIG. 4C, illustrates the case wherein some probes are shown hybridized with labeled targets under proper hybridization conditions.

FIG. 4D illustrates the case wherein maximum hybridization temperature is exceeded and no hybridization takes place.

### DETAILED DESCRIPTION OF THE INVENTION

Definitions and Overview:

Before describing the present invention in detail, it is to be understood that this invention is not limited to specific molecular probes, indicator materials or device structures, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a probe” includes a plurality of probes, reference to “an array” includes a plurality of arrays, reference to “a biomolecule” includes a combination of biomolecules, and the like.

In describing and claiming the present invention, the following terminology will be used in accordance with the definitions set out below.

The term “adsorb” as used herein refers to the noncovalent retention of a molecule by a substrate surface. That is, adsorption occurs as a result of noncovalent interaction between a substrate surface and adsorbing moieties present on the molecule that is adsorbed. Adsorption may occur through hydrogen bonding, van der Waal's forces, polar attraction or electrostatic forces (i.e., through ionic bonding). Examples of adsorbing moieties include, but are not limited to, amine groups, carboxylic acid moieties, hydroxyl groups, nitroso groups, sulfones and the like.

The term “array” used herein refers to a two-dimensional arrangement of features such as an arrangement of reservoirs (e.g., wells in a well plate) or an arrangement of fluid droplets or molecular moieties on a substrate surface (as in an oligonucleotidic or peptidic array). Arrays are generally comprised of regular, ordered features, as in, for example, a rectilinear grid, parallel stripes, spirals, and the like, but non-ordered arrays may be advantageously used as well. An array differs from a pattern in that patterns do not necessarily contain regular and ordered features. In addition, arrays and patterns of molecular probes of a substrate surface as provided herein are preferably substantially invisible to the unaided human eye. Arrays typically but do not necessarily comprise at least about 4 to about 10,000,000 features, generally in the range of about 4 to about 1,000,000 features.

The term “attached,” as in, for example, a substrate surface having a molecular moiety “attached” thereto (e.g., in the individual molecular moieties in arrays generated using the methodology of the invention) includes covalent binding, adsorption, and physical immobilization. The terms “binding” and “bound” are identical in meaning to the term “attached.”

The term “biomolecule” as used herein refers to any organic molecule, whether naturally occurring, recombinantly produced, or chemically synthesized in whole or in part, that is, was or can be a part of a living organism. The term encompasses, for example, nucleotides, amino acids and monosaccharides, as well as oligomeric and polymeric species such as oligonucleotides and polynucleotides, peptidic molecules such as oligopeptides, polypeptides and proteins, and saccharides such as disaccharides, oligosaccharides, polysaccharides, and the like.

It will be appreciated that, as used herein, the terms “nucleoside” and “nucleotide” refer to nucleosides and nucleotides containing not only the conventional purine and pyrimidine bases, i.e., adenine (A), thymine (T), cytosine (C), guanine (G) and uracil (U), but also protected forms thereof, e.g., wherein the base is protected with a protecting group such as acetyl, difluoroacetyl, trifluoroacetyl, isobutyryl or benzoyl, and purine and pyrimidine analogs. Suitable analogs will be known to those skilled in the art and are described in the pertinent texts and literature. Common analogs include, but are not limited to, 1-methyladenine, 2-methyladenine, N⁶-methyladenine, N⁶-isopentyl-adenine, 2-methylthio-N⁶-isopentyladenine, N,N-dimethyladenine, 8-bromoadenine, 2thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluoro-uracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “oligonucleotide” shall be generic to polydeoxynucleotides (containing 2-deoxy-D-ribose), to polyribonucleotides (containing D-ribose), to any other type of polynucleotide which is an N-glycoside of a purine or pyrimidine base, and to other polymers containing nonnucleotidic backbones, providing that the polymers contain nucleobases in a configuration that allows for base pairing and base stacking, such as is found in DNA and RNA. Thus, these terms include known types of oligonucleotide modifications, for example, substitution of one or more of the naturally occurring nucleotides with an analog, intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.). There is no intended distinction in length between the term “polynucleotide” and “oligonucleotide,” and these terms will be used interchangeably. These terms refer only to the primary structure of the molecule. As used herein the symbols for nucleotides and polynucleotides are according to the IUPAC-IUB Commission of Biochemical Nomenclature recommendations (Biochemistry 9:4022, 1970).

“Peptidic” molecules refer to peptides, peptide fragments, and proteins, i.e., oligomers or polymers wherein the constituent monomers are alpha amino acids linked through amide bonds. The amino acids of the peptidic molecules herein include the twenty conventional amino acids, stereoisomers (e.g., D-amino acids) of the conventional amino acids, unnatural amino acids such as a,oc-disubstituted amino acids, N-alkyl amino acids, lactic acid, and other unconventional amino acids. Examples of unconventional amino acids include, but are not limited to, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine.

The term “discrete” is typically used herein in its ordinary sense and refers to a region of a substrate that constitutes a separate or distinct part with respect to another region of the substrate. Thus, one discrete region of a substrate such as the interior region is readily distinguishable from another region such as the surface.

The term “fluid” as used herein refers to matter that is nonsolid or at least partially gaseous and/or liquid. A fluid may contain a solid that is minimally, partially or fully solvated, dispersed or suspended. Examples of fluids include, without limitation, aqueous liquids (including water per se and salt water) and nonaqueous liquids such as organic solvents and the like. As used herein, the term “fluid” is not synonymous with the term “ink” in that ink must contain a colorant.

The term “hybridizing conditions” is intended to mean those conditions of time, temperature and pH and the necessary amounts and concentrations of molecular moieties and reagents sufficient to allow at least a portion of a nucleotidic moiety to anneal with its complementary sequence. As is well known in the art, the time, temperature and pH conditions required to accomplish hybridization depend on the size or length of the oligonucleotide moiety to be hybridized, the degree of complementarity between the oligonucleotide probe and target, the presence of secondary structure in the probe and the target, and the presence of other materials in the hybridization reaction admixture. The actual conditions necessary for each hybridization step are well known in the art or can be determined without undue experimentation.

“Optional” or “optionally” means that the subsequently described circumstance may or may not occur, so that the description includes instances where the circumstance occurs and instances where it does not.

The term “probe” as used herein refers to a molecular moiety that exhibits a reaction in response to the presence of a “target.” Typically, the probes and targets are complementary to each other chemically so as to exhibit a “target-probe interaction.” Examples of target-probe interactions include hybridization reactions between nucleotidic moieties and antibody-binding reactions such as that exhibited when an antibody reacts with a particular protein or when an epitope reacts with a portion of a particular protein. Thus, two targets may represent different portions of a single molecule. For example, two different nucleotidic targets may represent two different sequenced portions of a single polynucleotide. In addition, probes are typically bound to a substrate while targets, if they are present in a fluid sample, are substantially suspended in the fluid sample.

The term “substrate” as used herein refers to any material having a surface onto which a probe may be bound and/or one or more fluids may be deposited. The substrate may be constructed in any of a number of forms such as disks, wafers, slides, well plates, membranes, for example. In addition, the substrate may be porous or nonporous as may be required for any particular fluid deposition. Suitable substrate materials include, but are not limited to, supports that are typically used for solid phase chemical synthesis, e.g., polymeric materials (e.g., polystyrene, polyvinyl acetate, polyvinyl chloride, polyvinyl pyrrolidone, polyacrylonitrile, polyacrylamide, polymethyl methacrylate, polytetrafluoroethylene, polyethylene, polypropylene, polyvinylidene fluoride, polycarbonate, divinylbenzene styrene-based polymers), agarose (e.g., Sepharose(®), dextran (e.g., Sephadex(®), cellulosic polymers and other polysaccharides, silica and silica-based materials, glass (particularly controlled pore glass, or “CPG”) and functionalized glasses, ceramics, and such substrates treated with surface coatings, e.g., with microporous polymers (particularly cellulosic polymers such as nitrocellulose), metallic compounds (particularly microporous aluminum), or the like. While the foregoing support materials are representative of conventionally used substrates, it is to be understood that the substrate may in fact comprise any biological, nonbiological, organic and/or inorganic material, and may be in any of a variety of physical forms, e.g., particles, strands, precipitates, gels, sheets, tubing, spheres, containers, capillaries, pads, slices, films, plates, slides, and the like, and may further have any desired shape, such as a disc, square, sphere, circle, etc. The substrate surface may or may not be flat, e.g., the surface may contain raised or depressed regions. Moreover, a portion of the substrate, discrete or otherwise, may be composed of data storage media for containing machine-readable information.

The term “surface modification” as used herein refers to the chemical and/or physical alteration of a surface by an additive or subtractive process to change one or more chemical and/or physical properties of a substrate surface or a selected site or region of a substrate surface. For example, surface modification may involve (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

The Inventive Device:

Overall, the present invention involves devices having at least one indicator to record conditions to which the device has been exposed. The devices are typically used in molecular or biomolecular assays wherein probes of the device interact with targets that may be present in a sample, and the indicators provide a record of the assay conditions. The record of the assay conditions provides users of the inventive device information useful in assay result analysis. That is, if probe-target interactions are observed, the record of the assay condition is useful in interpreting the significance of such interactions.

In one embodiment, the invention pertains to a device comprising a substrate having a plurality of different molecular probes attached to a surface thereof and an integrated indicator that exhibits a response when exposed to a condition to which the substrate may be exposed. Each of the different molecular probes is selected to interact with a different corresponding target, and the indicator response is detectable after the indicator is removed from the condition. Typically, the indicator response to the condition is detectable for at least 1 minute after removing the indicator from the condition. The indicator response is preferably detectable for at least 1 hour, and, optimally, the indicator response is substantially and permanently detectable after removing the substrate from the condition.

The indicator may be responsive to a condition that facilitates, enhances, hinders or prevents target-probe interaction. For example, the condition may be an environmental condition that may or may not be predetermined to affect target-probe interaction. Such an environmental condition may be a maximum temperature, a minimum temperature or a temperature range. Other examples of environmental condition include, but are not limited to, water content, chemical content and chemical concentration. The indicator response may be optically, magnetically and/or electrically detectable, optionally by a machine. The response may occur after exposure of the indicator to the condition for at least a predetermined period. A predetermined period is typically about 1 minute to about 48 hours, preferably under 24 hours, e.g., about 5 to about 10 hours and optimally about 6 to about 8 hours.

The molecular probes may be comprised of any chemical moiety that allows interaction with a corresponding target. For example, when the targets are biomolecular, it is preferred that the molecular probes are biomolecular as well. Such probes may be nucleotidic, peptidic, oligomeric or polymeric. The targets may be single molecules, represent portions of a single molecule, or portions of a single cell. In addition, it is preferred that the molecular probes are arranged in an array on the substrate surface. Irrespective of whether the probes are arranged in an array, the arrangement should comprise at least about 10 probes typically per square centimeter of substrate surface. Typically, at least about 50,000 probes are attached per square centimeter of surface. More preferably, at least about 200,000 probes/cm² are attached. Optimally, the substrate has attached thereto at least 1,000,000 probes/cm². Such probes may be attached or synthesized using acoustic ejection. While it is difficult with current technology to produce a substrate having a probe density of greater than about 2,000,000 probes/cm², it is envisioned that future probe densities will be limited predominantly by the size of the probes rather than production technology.

The substrate of the device may take a number of forms. For example, the substrate may comprise a disk, tape, well plate, a slide, or other objects commonly used as a substrate. Optionally, the substrate may further contain machine-readable information and/or a medium on which information may be written. Such medium is typically selected to contain electronic information and may be noncoplanar with respect to the surface on which the molecular probes are attached. Optimally, the medium is writable from a surface that opposes the surface on which the molecular probes are attached. Devices comprising a substrate having molecular moieties attached to a surface thereof and containing machine-readable information are described in U.S. patent application Ser. No. ______, (“Integrated Device with Surface-Attached Molecular Moieties and Related Machine-Readable Information”), inventors Ellson, Foote and Mutz, filed on Nov. 13, 2000 and assigned to Picoliter, Inc. (Cupertino, Calif.).

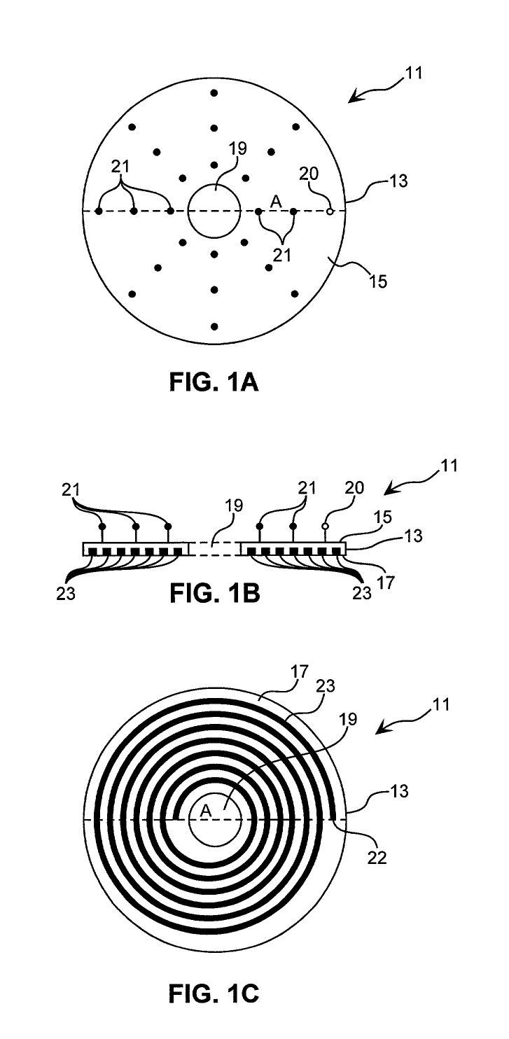

FIG. 1 schematically illustrates an example of the above-described embodiment wherein the substrate is in the form of a disk, specifically a compact disk. As with all figures referenced herein, in which like parts are referenced by like numerals, FIG. 1 is not necessarily to scale, and certain dimensions may be exaggerated for clarity of presentation. The device 11 is comprised of a solid circular disk 13 having opposing and substantially parallel surfaces, indicated at 15 and 17, respectively. Located at the center of the disk is a circular hole 19 extending through the disk. Attached to exterior surface 15 is a plurality of different molecular probes 21 in the form of an array. That is, the molecular probes 21 represent features of the array, with the features forming concentric circles about the center hole 19 of the disk. As such, the disk is substantially symmetric about its center and is thus substantially rotationally uniform. Preferably, the radial mass distribution of the disk is also substantially uniform. Other distributions of probes are possible provided that the mass distribution does not substantially interfere with the rotational stability of the disk. Rotational stability depends on mass distribution, rate of rotation, and other parameters for disk and rotational means design known in the art. Each of the different molecular probes 21 is selected to interact with a different corresponding target. Thus, by determining which probes exhibit evidence of a response from interaction with a target, a sample that may contain targets may be assayed.

Also shown on surface 15 is an integrated indicator 20 that exhibits a response after exposure to a condition to which the disk 13 may be exposed. The indicator response, if triggered, indicates whether the disk has been exposed to the condition. Thus, the presence or absence of the indicator response may be used as a quality control measure to assess the accuracy and/or reliability of the assay. Because the integrated indicator is on the same surface as the molecular probes 21, a detector for detecting probe-target interaction may also be adapted to detect the indicator response as discussed below. In addition, when the substrate is symmetrical, axial or otherwise, it is useful to establish the orientation of the substrate with respect to the detector. Thus, either or both of surfaces 15 and 17 may be marked to establish orientation. For example, a reference molecular moiety may be used to establish a reference point on the surface to which the probes are attached. As shown, the integrated indicator 20 itself serves as such a reference point.

Optionally, the disk contains a medium on which information may be written. Typically, such medium is contained in a discrete portion of the device. As shown, the medium is contained in the disk 13 as a spiral track 23. One way in which information may be written on the medium is to encode data as a series of reflective features and non-reflective pits. In such a case, the information is optically readable by rotating the disk 13 about the center hole 19 and providing an optical reader adapted to read the information from the underside 17 of the disk 13. Design and construction of such optical readers are well known in the art. As the information is located within the disk as a spiral track 23 rather than on the surface 15 to which the molecular probes 21 are attached, it is evident that the information is located in a discrete region of the disk that is noncoplanar with respect to surface 15 on which the molecular probes 21 are attached. In this case, it is desirable to ensure that a spatial correspondence between the information contained in the disk and the probes attached to the disk. Thus, the integrated indicator 20 may be located at the nearest point on surface 15 to the location of end 22 of the spiral track 23. This allows the reading of machine-readable information to act as a positional encoder for properly depositing the moieties on the opposing surface. In other words, the act of reading the machine-readable information from the spiral track 23 on surface 17 could determine the rotational position of the disk 13. This correspondence may be used to improve the timing of release of materials by a deposition system adapted for controlled delivery of materials to the substrate.

In another embodiment, the invention pertains to a device comprising a substrate having a plurality of molecular probes attached to a surface thereof and a plurality of different integrated indicators. Each indicator is selected to exhibit a response when exposed to one of a plurality of conditions to which the substrate may be exposed. The molecular probes are selected to interact with corresponding targets. The indicator response is detectable after removing the indicator from the condition.

This embodiment is similar to the above-described embodiment in that it provides for an indicator response to a condition, wherein the response is detectable as described above. The molecular probes of this embodiment and the arrangement thereof are also described above. Furthermore, the substrate of this embodiment may also generally take the forms of the previously described embodiment. This embodiment, however, provides for a greater amount of information relating to the exposure of device and associated probes to various conditions. Such additional information may in turn, ensure the accuracy of assays carried out using the probes of the substrate. For example, the molecular probes of this embodiment may be selected to interact with corresponding targets when exposed to at least one of the plurality of conditions. Alternatively, the molecular probes may be selected to interact with corresponding targets when exposed to all of the conditions. Optimally, the molecular probes may be selected to interact with corresponding targets when exposed to all of the conditions simultaneously

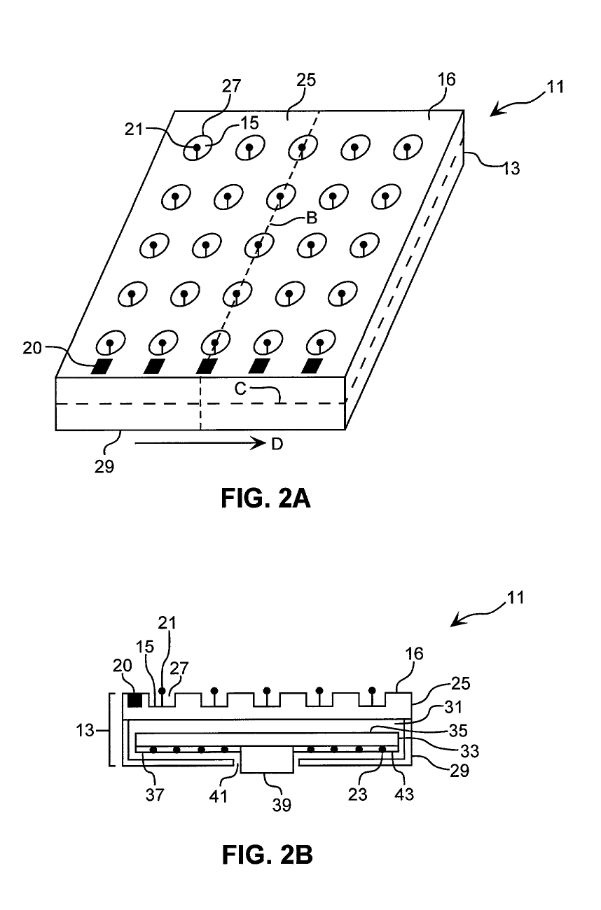

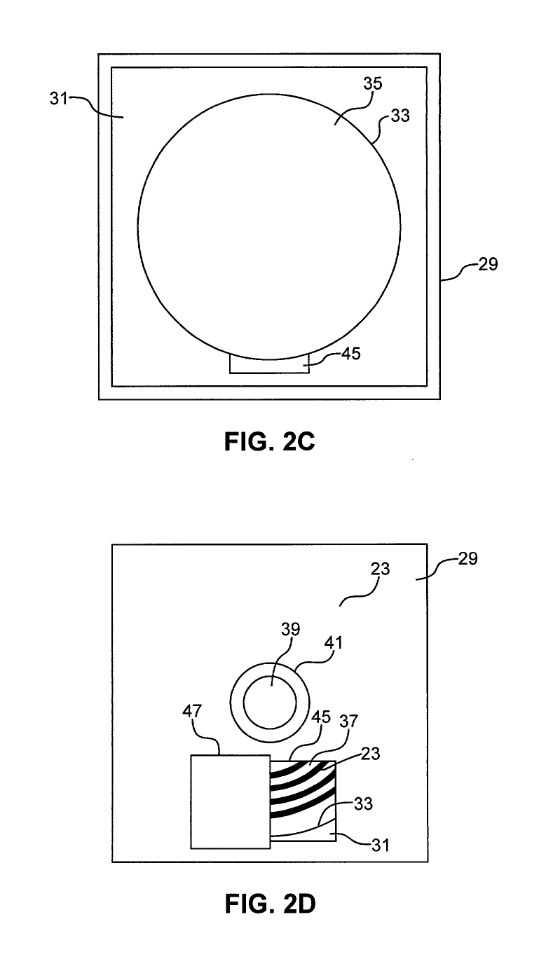

FIG. 2 schematically illustrates an example of the above-described embodiment wherein the substrate is in the form of a well plate. The device 11 is comprised of a well plate 13 having individual wells 27 terminating at openings in an exterior surface 16 and arranged in an array. Such well plates are commercially available from Corning Inc. (Corning, N.Y.) and Greiner America, Inc. (Lake Mary, Fla.). As shown, each individual well 27 has a molecular probe 21 bound to an interior surface 15 thereof. However, the probe is not necessarily covalently bound to the plate. For example, the probe may be in solution. As a general rule, though, if an array of probes is located in an interior surface of the well, the probe is bound to the surface.

In addition, an array of integrated indicators 20 is also provided on the exterior surface of the well plate 13. As shown, the indicators 20 are placed in a row on a portion of the exterior surface 16. As described above, these indicators 20 exhibit a response after exposure to a condition to which the well plate 13 may be exposed. These indicators may each indicate a different condition, or some may indicate the same condition. For example, the indicators may each indicate a different temperature and arranged in order of increasing temperature in the direction indicated by Arrow D. In the alternative, any two of the indicators may be provided to indicate the same condition to ensure that each indicator has an auxiliary in case of failure. Due to the proximity of the indicators to the moieties, the indicators experience similar conditions as that experienced by the probes. As a result, the conditions indicated by the indicators closely approximate that experienced by the probes.

Optionally, the well plate 13 is attached to a cartridge base 29 to define a cartridge interior 31. A magnetic disk 33 is generally interposed between well plate 13 and the cartridge base 29 within the cartridge interior 31. The disk 33 is a generally flat and circular piece having an upper surface 35 and a lower surface 37. A cylindrical hub 39 extends perpendicularly from the center of the lower surface 37 of the disk 33 through circular opening 41 of the cartridge base 29. The disk is free to rotate about its hub in a generally free-floating manner. The lower surface 37 is coated with magnetic storage medium 43 that allows a spiral track 23 to be formed therein to magnetically store machine-readable information related to the molecular probes. Also optionally located in the cartridge base 29 is a rectangular opening 45 that provides external access to the magnetic disk contained in the cartridge interior 31. A slidable spring-loaded panel 47 covers the opening 45 in order to protect the magnetic medium on the disk from damage when the disk is not in use. As shown, the slidable panel 47 is positioned such that it does not cover the opening, thereby providing a magnetic reader access to the magnetic medium on the disk. Thus, the information contained in the spiral track 23 is ready for reading by a magnetic reader. Design, construction and use of such magnetic readers are well known in the art. For example, the magnetic reader may engage the disk by gripping the portion of the hub 39 that is accessible to the exterior to the cartridge and spinning the disk. This allows information contained in the spiral track to be read. As the information relating to the attached probes is located within the disk as a spiral track 23 rather than on the interior surfaces 15 of the well plate to which the molecular probes 21 are attached, it is evident that the information is located in a discrete region of the disk that is noncoplanar with respect to the surfaces 15. Optionally, one or more of the interior surfaces 15 may be covered with a protective layer (not shown) that protects the probes from damage as a result of improper handling. Devices for sealing well plates are commercially available from many sources including TekCel Corporation (Hopkinton, Mass.). Such protective coatings may also be adapted to protect the integrated indicators.

In still another embodiment, the invention pertains to a device comprising a substrate having a plurality of nucleotidic molecular probes attached to a surface thereof and an integrated indicator that exhibits a response when exposed to a condition to which the substrate may be exposed. The nucleotidic molecular probes are selected to interact with corresponding targets. This embodiment also provides for an indicator response to a condition wherein the response is detectable as described above. The nucleotidic molecular probes of this embodiment and the arrangement thereof are also described above. Furthermore, the substrate of this embodiment may also take the forms of the previously described embodiment. This embodiment, however, provides a device that is especially useful in determining the nucleotidic content of a sample when the condition represents a hybridization condition between the probes and targets.

For example, the nucleotidic molecular probes of this embodiment may be selected to interact with corresponding targets when exposed to at least one of the plurality of conditions. Alternatively, the molecular probes may be selected to interact with corresponding targets when exposed to all of the conditions. Optimally, the molecular probes may be selected to interact with corresponding targets when exposed to all of the conditions simultaneously.

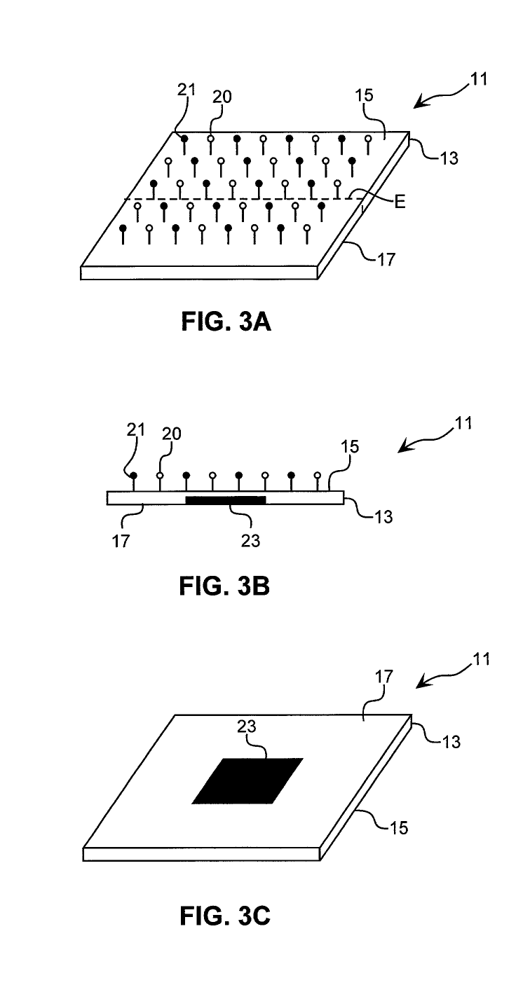

FIG. 3 schematically illustrates in simplified cross-sectional view another version of the inventive device. This version uses an ordinary microscope slide as the substrate. The device 11 is comprised of a rectangular slide 13 having opposing and substantially parallel surfaces, indicated at 15 and 17, respectively. The slide may be formed in any convenient size, but is preferably a solid support such as a standardized glass microscope slide that has a rectangular surface of about 3 inches by 1 inch (75 mm×25 mm). Optionally, the slide may have coatings of substantially uniform thickness applied to various portions of its surface to form a raised exterior surface to improve the attachment of probes or indicators. Attached to exterior surface 15 is a plurality of nucleotidic molecular probes 21 in the form of an array. That is, the nucleotidic molecular probes 21 represent individual features of the array, with the features forming a preferably rectilinear array such that each feature has four nearest neighbors, each spaced the same distance apart.

While only one indicator is required for this embodiment, an array of integrated indicators 20 is shown provided on exterior surface 15 of the slide 13. As shown, the indicators 20 are also placed in a rectilinear array, wherein each indicator is located adjacent to a probe. That is, the indicators are uniformly interspersed among the nucleotidic molecular probes. As discussed above, these indicators exhibit a response after exposure to a condition to which the slide may be exposed. Interspersion among the probes also allows the indicators to be exposed to substantially the same conditions as the nucleotide probes. As such, if a sample is applied to the probes for assaying nucleotidic content of the sample, the indicators should provide an accurate measure of whether the hybridization conditions are met.

Optionally, information relating to the molecular probes is contained in an electronic microchip 23 that provides sufficient memory to store such information. As shown, the microchip 23 is embedded in the slide 13. Such a microchip 23 may be partially exposed at surface 17, as shown in FIG. 3, or located entirely within the substrate. Such microchips are often employed in smart cards, e.g., plastic cards resembling a credit card that contains a computer chip, which enables the holder to perform various operations, such as mathematical calculations, paying of bills, and the purchasing of goods and services. Use of smart card technology in conjunction with nucleotidic probes is described in U.S. Ser. No. ______ (“Integrated Device with Surface-Attached Molecular Moieties and Related Machine-Readable Information”), inventors Ellson, Foote and Mutz, filed on Nov. 13, 2000 and assigned to Picoliter, Inc. (Cupertino, Calif.).

As discussed above, any of the indicators for use in the invention may be responsive to various predetermined or other conditions such as conditions that facilitate, enhance, hinder or prevent target-probe interaction. Thus, the indicator should be selected to exhibit a response that indicates with sufficient accuracy and precision whether target-probe interaction conditions are met. Preferably, the response is sufficiently similar to the target-probe interaction signal such that both can be detected using the same detection means. For example, nucleotidic probe-target interactions such as hybridization are often detected through fluorescence readers, such as the GenePix 4000 from Axon Instruments, Inc. (Foster City, Calif.). Condition indicators employed in hybridization arrays may therefore also exhibit fluorescent responses to hybridization. Similarly, indicators capable of generating signals having a form other than fluorescence may also indicate a condition to which the substrate may be exposed, if the target-probe response exhibits the same form of signal.

The Environmental Condition Indicators:

An important environmental condition for hybridization as well as other biomolecular assays is temperature. Various temperature indicators are known in the art to respond to temperatures through dimensional and/or chromatic changes. For example, wax shapes having specific melting points can be used to indicate temperatures to which the shapes have been exposed. In addition, when nucleotidic or other types of probes are employed in the inventive device, nucleotidic indicators may also be employed to indicate temperatures to which the substrate has been exposed. For example, it is known that double-stranded DNA dissociates to single strands at a temperature that depends on its nucleotide content. It is also known that G-C base pairs are bound by three hydrogen bonds and hence dissociate at a higher temperature than A-T base pairs, which employ two hydrogen bonds. The temperature at which a particular sample of DNA is 50% dissociated into single strands is known as the melting temperature (Tₘ). Tₘ is very sensitive to the specific sequences of associating DNA pairs. It should be evident from the above disclosure that this phenomenon may be exploited to produce nucleotidic temperature indicators having a predetermined Tₘ. In order to ensure that the indicators exhibit a specific Tₘ, one may produce such nucleotidic temperature indicators by controlling the composition, sequence and length of the oligonucleotides or polynucleotides that form the indicators.

For example, one or more nucleotidic features may be used as temperature indicators in the present invention. Such features may contain either single-stranded oligonucleotides having defined sequences prehybridized to a labeled target, or double-stranded oligonucleotides having one labeled strand. In either case, the non-labeled strand is attached to the substrate. Typically, the attached strand is longer than the labeled strand, leaving the shorter, labeled strand free to dissociate from the long strand when Tₘ is reached. Thus, when the inventive device is subjected to an assay temperature, the labeled strand of the nucleotidic indicators having a Tₘ lower than the assay temperature would be released through melting, while indicators having a Tₘhigher than the assay temperature would retain the labeled strand. Accordingly, identifying the indicators that have melted and their associated Tₘ can determine the assay temperature.

It is important to ensure that melting does not release labeled strands that subsequently reattach to the device or rehybridize with a portion thereof, thereby resulting in a spurious signal or interfering with experimental data. Thus, employing melting nucleotides to monitor temperature may require providing a wash step to carry away strands released from such melting. In addition, other techniques may be employed to further mitigate such potentially deleterious effects. For example, indicators may be positioned on a discrete portion of the substrate to ensure that released label strands do not come into contact with the probes attached to the substrate. As another example, highly artificial or exotic sequences could also reduce the likelihood of interference with the probes. See, e.g., U.S. Ser. No. 09/669,267 (“Arrays of Oligonucleotides Containing Nonhybridizing Segments”), inventor Ellson, filed on Sep. 25, 2000 and assigned to Picoliter, Inc. (Cupertino, Calif.). As still another embodiment, a different label may be used with the temperature indicator nucleotidic material than that employed for the probe. Other ways to reduce such interference may be known in the art as well.

Complementary single-stranded nucleotidic material can be annealed to induce hybridization, and this phenomenon is the basis of all nucleic acid hybridization technology. The kinetics of single strand association is second order and necessarily more complex than that of dissociation, depending on the relative concentration of the components as well as factors such as degree of sequence repetition, ionic strength, pH and temperature. Thus, this annealing phenomenon can also be employed to provide an indication of temperature as well as other environmental conditions relating to hybridization such as pH.

One way in which annealing may be used to measure temperature during a hybridization assay is to employ a series of features as indicators, each feature containing a plurality of single-stranded nucleotidic oligomers. Each feature contains random-sequence oligonucleotidic strands having the same ratio of triple-hydrogen-bond bases to double-hydrogen-bond bases. That is, the ratio of Gs and Cs to As and Ts is the same for all nucleotidic strands within a feature. When a sample is applied to both device probes and indicator oligonucleotides, a portion of the indicator strands and labeled targets in the sample may bind. For a given assay, hybridization kinetics might be expected to favor features containing oligonucleotides with a Tₘ above the assay temperature. Therefore, with proper calibration, hybridization would be detected at high Tₘ features and be absent from low Tₘ features. A gradient or cutoff of signal intensity could then be used to determine the temperature at which the hybridization was performed.

A potential problem with the above annealing approach is that positional effects and degree of sequence repetition can also affect hybridization kinetics. Thus, random-sequence strands of a feature may exhibit a range of hybridization rates, even if the triple-hydrogen-bond to double-hydrogen-bond base ratio for the strands are identical. This variability complicates the precise determination of the temperature from mere observing of hybridization activity of the features. Instead of using randomly sequenced strands as temperature indicators, another approach is to employ strands having sequences corresponding to well-characterized sequences known to be present in the sample, e.g., sequences associated with a housekeeping gene. This approach tends to increase signal intensity associated with hybridization.

Still another hybridization approach is similar to the above, except that the temperature indicator strands are keyed to calibrant strands incorporated into the sample. It is preferable that the calibrant strands contain highly artificial or exotic sequences to reduce the likelihood of binding with the probes and thereby interfering with the desired target-probe interactions. This approach has the advantage that both temperature indicator strands and calibrant strands can be carefully controlled to indicate precise assay conditions.

For hybridization assays, the indicator may be employed to indicate a predetermined temperature associated with hybridization. For example, the predetermined temperature may be a maximum hybridization temperature of about 60° C. to about 90° C. for hybridization. In the alternative, the predetermined temperature may be a minimum hybridization temperature of about 35° C. to about 45° C. The precise predetermined temperature will vary according to the precise nature of the assay. These temperature ranges may be useful for non-hybridization assays as well.

In addition to temperature, there are other environmental conditions that affect hybridization and other biomolecular assays. These conditions include, for example, water content and chemical concentration. Thus, the indicators of the present invention may be chosen to provide a record of these conditions as well. For example pH sensitive compounds are well known in the art and a number of references disclose their incorporation in substrates. In a preferred embodiment, the indicator is also surface bound and exhibits a fluorescent response to pH. Offenbacher et al. (1986), “Fluorescence Optical Sensors for Continuous Determination of Near Neutral pH Values,” Sensors and Actuators 9: 73-84, reports that glass-immobilized sensors may allow for pH determination in the range of 6.4 to 7.7. That is, a high-sensitivity, fluorescence-based pH probe such as 7-hydroxycoumarin-3-carboxylic acid can be embedded in glass by coupling to surface amines by the commonly used coupling reagent 1-ethyl-3-(3-dimethylaminoproyl)-carbodiimide hydrochloride. Such probes have different protonation states in response to local environmental pH. In addition, these differing protonation states translate into different intensities of emission spectra and can be used to calibrate a pH determination to a standard deviation of about ±0.01 unit.

To create an effective indicator for ionic strength, one may hydroxylate the glass surface and introduce ionic sensitivity to the fluorescent readout. The presence of hydroxyl groups in the proximity of the fluorescent indicators induces electrostatic interaction between the charged groups, and the surface can then serve as a readout of ionic strength. Ionic strength effects the dissociation constant of weak electrolytes such as 1-hydroxypyrene-3,6,8-trisulfonate or as 7-hydroxycoumarin-3-carboxylic acid according to equation (I):

pKₐ^{I}^{=pK}ₐ^{TH}+(0.512z_{B}²−zH_{B}²)I^{½}/(1+1.6I^{½}) (I)

where pKₐ^{I} represents the acid dissociation constant at ionic strength I, pKₐ^{TH} is the thermodynamic acid dissociation constant, and ZB and ZHB are the charges of the deprotonated and protonated species, respectively. See Wolfbeis et al., (1986) “Fluorescence Sensor for Monitoring Ionic Strength and Physicological pH Values,” Sensor and Actuators, 9:85-91.

As another example, Obnik et al, (1998) “pH Optical Sensors Based on Sol-Gels: Chemical Doping versus Covalent Immobilization” Analytica Chimica Acta 367: 159-165, reports the use of silica-immobilized aminofluorescein to detect changes in the pH range 4 to 9. Through use of a flow cell, pH changes are reported as changes in fluorescence signal intensity (max emission wavelength varies with conditions) after excitation at 490 nm. Aminofluorescein is described as either covalently bound to, or doped into a silica sol-gel which, in turn, is fixed onto a glass slide as a thin layer. It is further disclosed that covalent binding provides superior stability, while doping affords an easier and more general synthesis.

As a further example, Wolfbeis et al., (1992) “LED-Compatible Fluorosensor for Measurement of Near-Neutral pH Values,” Mikrochimica Acta 108:133-141 reports that the fluorescent indicator 5-(and 6-)carboxynaphthofluorescein can be immobilized and used in a pH range of 6 to 9. This indicator can be immobilized in two ways, either by covalent attachment to a cellulose matrix, or by physical entrapment in a sol-gel glass. The cellulose conjugate can be formed into sensing membranes of approximately 30 microns thick, while the sol-gel was deposited onto glass slides as in the previous reference. Again, detection is carried out in a flow cell. Spectral characteristics varied between the various conditions. Interestingly, by using an excitatory wavelength above 500 nm, the sensors are compatible with conventional LEDs. The cellulose formulation is considered superior for constructing a pH sensor, because of the stability of the covalent fluorophore immobilization. It should be apparent, then, that any of these sensors and immobilization techniques can be incorporated into the present invention to form the above-described integrated indicators. Such indicators employed in the present invention typically respond to a pH of about 5 to about 9.

Similarly, salinity indicators, e.g., compounds that are sensitive to sodium and/or chloride, as well as formamide concentration indicators are also known in the art. Typically salinity indicators of the present invention are responsive to a salinity of about 0.01 molar to about 8 molar. The indicators may be employed to detect the presence or concentration of a chemical moiety that either enhances or hinders target-probe interactions.

It should be noted that certain condition indicators, notably pH sensitive indicators, exhibit a response when exposed to the condition but revert to their original state soon after removal from the condition. Thus, preferred indicators exhibit substantially irreversible responses rather than reversible response. If reversible, the response reversal preferably takes an extended amount of time after removal from the condition that triggers the response to allow the response to be recorded in a more permanent form of information, e.g., by writing to an information-storage medium on the device. In short, when the indicator response is reversible, one of ordinary skill in the art will recognize that the response may be converted into a permanent form before complete reversal.

Method and Apparatus for Assaying a Sample Using the Inventive Device:

In another embodiment, the invention provides a method for assaying a sample using any of the above devices. The sample is exposed to an assay condition by contacting the molecular probes attached to the substrate surface of the device. Then, the indicator is examined to determine whether the assay condition has triggered the indicator response to the condition. Depending on whether the indicator response is detected, the probe-target interactions are assessed. For example, if an indicator is chosen to respond only to appropriate assay conditions, then probe-target interaction is assessed when the indicator response is detected. Similarly, if an indicator is chosen to respond only to inappropriate assay conditions, then assessment of the probe-target interaction is likely also inappropriate.

Depending on the desired probe-target interaction, assays must be adjusted accordingly. For example, for nucleotidic probe-target interactions, it is generally desirable to maintain conditions for hybridization assays by placing the sample and the device in a controlled environment, heating the device while the sample is in contact therewith and preventing the sample from evaporating. After hybridization but before detection for hybridization, excess sample is typically removed from the device. In the case wherein the indicator is also nucleotidic, detection of the probe-target interaction and of the indicator response is preferably carried out using a single reader. When the inventive device contains a medium on which information may be written, it may be desirable to record whether the response and/or the probe-target interaction occurred as information contained in the device.

The assay method may be carried out by employing an apparatus for assaying a sample using the inventive device. The apparatus comprises an applicator for applying a sample to the molecular probes and an indicator-response detector for detecting whether any of the indicators of the inventive device exhibit a response. Typically, the apparatus further includes an interaction detector for detecting probe-target interactions. Such an interaction detector may be a known or yet to be developed optical, magnetic or electric detector. Depending on the type of indicator of the device, the interaction detector may be activated or deactivated when the indicator-response detector detects a response by the indicator. Optimally, the indicator-response also serves as an interaction detector for detecting probe-target interactions.

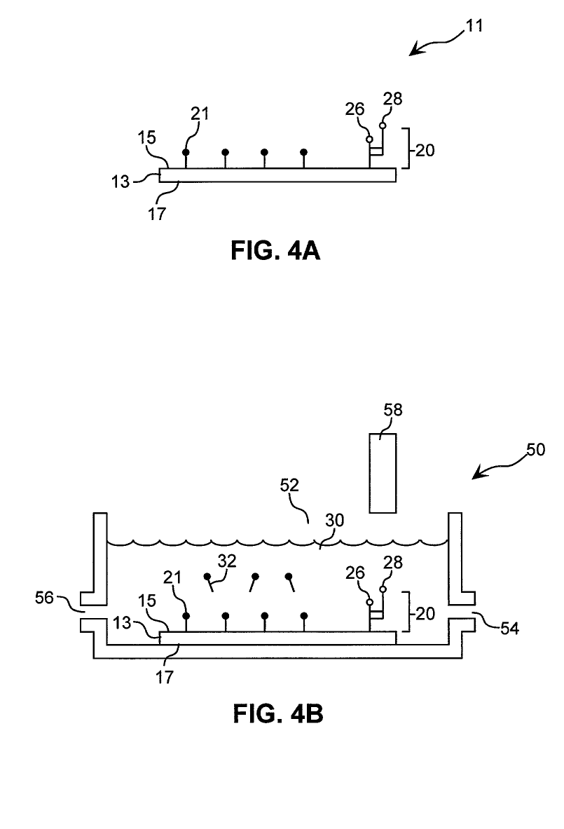

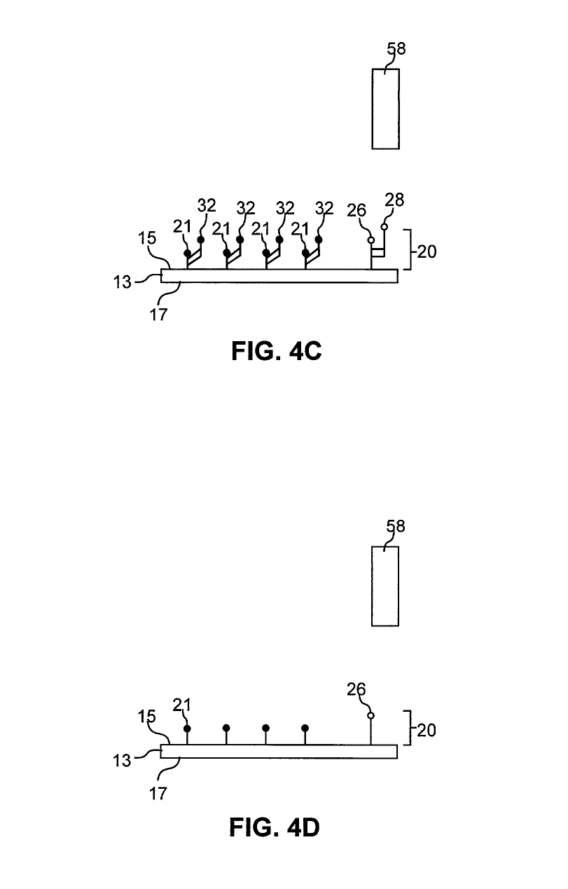

FIG. 4 illustrates an example of the above described method and apparatus for assaying a sample using the inventive device similar to that illustrated in FIG. 3. While this example illustrates a nucleotidic assay, it should be evident that one of ordinary skill in the art may modify the disclosure of this example to carry out other types of assays, e.g., peptidic and other biomolecular assays. As shown in FIG. 4A, a device 11 is provided that is similar in construction to the device illustrated in FIG. 3 comprising a rectangular slide 13 having opposing and substantially parallel surfaces, indicated at 15 and 17, respectively. Attached to exterior surface 15 is a plurality of different nucleotidic molecular probes 21 in the form of an array, each different nucleotidic molecular probe selected to hybridize with a different corresponding nucleotidic target. Also shown on surface 15 is an integrated indicator 20 comprising a number of identical double-stranded oligonucleotides having a Tₘ equal to the maximum temperature under which the probes 21 will properly hybridize with their corresponding nucleotidic targets. For simplicity, only one double-stranded oligonucleotide is shown representing the indicator comprising one nucleotidic strand 26 attached to the substrate and one fluorescently labeled nucleotidic strand 28 is hybridized with strand 26.

As shown in FIG. 4B, the device 11 is then loaded into a hybridization chamber 52 of an apparatus 50 for assaying a sample using the inventive device 11. It should be apparent that the hybridization chamber should produce conditions that are suitable for hybridization such as providing heat, preventing sample evaporation, and performing other tasks associated with the assay. The chamber is filled from an inlet 54 with a fluid sample 30 that contains fluorescently labeled nucleotidic targets 32 that may or may not hybridize with the probes 21 attached to the slide 13, thereby submerging the device. As a result, the fluid sample 30 comes into contact with the probes 21. The chamber is then closed and brought to assay conditions while the apparatus 50 moves the fluid sample 30 and/or device 11 to ensure proper fluid contact with the probes of the device within the chamber 52. After sufficient time has passed, the fluid is drained from the hybridization chamber 52 out of outlet 56, an optional wash step is carried out to remove nonhybridized labeled targets from the slide surface 15. Then, a fluorescence detector 58 of the apparatus 50 is employed to detect whether the indicator 20 exhibits fluorescence.

In the case where assay conditions are appropriate for hybridization and targets corresponding to the probes are present in the sample, the probes 21 will hybridize with the targets. This is the case shown in FIG. 4C, wherein some probes are shown hybridized with labeled targets. Since assay conditions never exceeded the maximum hybridization temperature of the probes 21, the two nucleotidic indicator strands 26, 28 remain hybridized. Thus, the fluorescence detector 58 will detect the presence of fluorescently labeled indicator strand 28 and proceed to detect for the target-probe interactions by detecting for fluorescent at the probes 21. However, if the assay conditions are such that the assay temperature exceeds Tₘ, then the fluorescent detector 58 should detect little or no fluorescence at the indicator 20 since the labeled strand 28 has melted away. In such a case, as shown in FIG. 4D, there may be no detectable fluorescence at the probes 21. In addition, even if fluorescence is detected at the probes, such fluorescence may not indicate hybridization, only the presence of fluorescent labels. Thus, in this case, absence of fluorescence at the indicator indicates assay conditions inappropriate for hybridization. It should be evident that the above-described apparatus or any portion thereof may employ electromechanical and/or computerized components to carry out the desired assay.

Precursor to the Inventive Device:

In another embodiment, the invention pertains to a device comprising a substrate having a surface adapted for attachment to a plurality of molecular moieties. An integrated indicator is included in the device and exhibits a response when exposed to a condition. The response is detectable after removing the indicator from the condition. Typically, response indicates whether the substrate has been exposed to a condition that allows for or precludes attaching the plurality of molecular moieties to the substrate surface. This embodiment represents a precursor to the previously described embodiment illustrated in FIGS. 1-3. That is, by attaching a plurality of molecular moieties to the surface adapted for such a purpose, the previously described embodiments may be formed. This embodiment is particularly useful for ensuring that the molecular moieties are properly attached to the substrate surface.

Attachment of molecular moieties may be accomplished by using an apparatus for attaching molecular moieties to the substrate surface of the inventive device. A preferred apparatus is described in U.S. patent application Ser. No. 09/669,996, referred above. Such an apparatus enables preparation of molecular arrays, particularly biomolecular arrays, to order having densities allowed by the technology used to produce the arrays such as photolithographic processes, piezoelectric techniques (e.g., using inkjet printing technology), and microspotting. When focused acoustic energy is used, the array densities that may be achieved using the devices and methods of the invention are at least about 50,000 biomolecules per square centimeter of substrate surface, preferably at least about 200,000 per square centimeter of substrate surface. The biomolecular moieties may be, e.g., peptidic molecules and/or oligonucleotides.

Thus, such an apparatus for attaching molecular moieties to the substrate surface of the device as described above may comprise an indicator-response detector for detecting whether the indicator exhibits the response to the condition and a means for attaching a plurality of molecular moieties to the surface of the substrate. The attaching means may be activated if the indicator-response detector detects the response to the condition that allows for attaching the plurality of molecular moieties to the substrate surface. That is, a plurality of molecular moieties is attached to the substrate surface if the integrated indicator of the device exhibits a response that allows for attachment to the surface. Alternatively, if the occurrence of a response indicates a condition that precludes the attachment of the moieties to the substrate surface, a plurality of molecular moieties may be attached to the substrate surface if the integrated indicator does not exhibit a response to the condition. Various attachment methods are disclosed, e.g., in U.S. patent application Ser. No. 09/669,996. It should be noted that such an apparatus may be employed to attach molecular probes as well as indicators to the substrate of the inventive device.

The chemistry employed in synthesizing substrate-bound oligonucleotides in this way will generally involve now-conventional techniques known to those skilled in the art of nucleic acid chemistry and/or described in the pertinent literature and texts. See, for example, DNA Microarrays: A Practical Approach, M. Schena, Ed. (Oxford University Press, 1999). That is, the individual coupling reactions are conducted under standard conditions used for the synthesis of oligonucleotides and conventionally employed with automated oligonucleotide synthesizers. Such methodology is described, for example, in D. M. Matteuci et al. (1980) Tet. Lett. 521:719, U.S. Pat. No. 4,500,707 to Caruthers et al., and U.S. Pat. Nos. 5,436,327 and 5,700,637 to Southern et al.

Alternatively, an oligomer may be synthesized prior to attachment to the substrate surface and then “spotted” onto a particular locus on the surface using the methodology of the invention as described in detail above. Again, the oligomer may be an oligonucleotide, an oligopeptide, or any other biomolecular (or nonbiomolecular) oligomer moiety. Preparation of substrate-bound peptidic molecules, e.g., in the formation of peptide arrays and protein arrays, is described in co-pending patent application U.S. Ser. No. 09/669,997 (“Focused Acoustic Energy in the Preparation of Peptidic Arrays”), inventors Mutz and Ellson, filed on Sep. 25, 2000 and assigned to Picoliter, Inc. (Cupertino, Calif.). Preparation of substrate-bound oligonucleotides, particularly arrays of oligonucleotides wherein at least one of the oligonucleotides contains partially nonhybridizing segments, is described in co-pending patent application U.S. Ser. No. 09/669,267 (“Arrays of Oligonucleotides Containing Nonhybridizing Segments”), inventor Ellson, also filed on Sep. 25, 2000 and assigned to Picoliter, Inc. (Cupertino, Calif.). In any case, attachment of an oligomer to a surface may involve surface modification in order to promote surface-probe adsorption or another type of attachment as discussed in U.S. Ser. No. ______, (“Integrated Device with Surface-Attached Molecular Moieties and Related Machine-Readable Information”), inventors Ellson, Foote and Mutz, filed on Nov. 13, 2000 and assigned to Picoliter, Inc. (Cupertino, Calif.).

Thus, the invention provides advantages previously unknown in microarray technologies. As discussed above, the invention may be used to avoid the problems associated with labeling samples with different colored tags while exhibiting improved performance. In addition, if two substantially identical inventive devices are employed in hybridization assays but at different times and/or locations, the inventive device may be used to determine whether experimental conditions are the same between the assays. Thus, it should be evident that the device may be used to reduce experimental error arising from using different equipment such as hybridization chambers produced by different manufacturers. Moreover, the devices can be used to determine the optimal range of conditions for particular assays. For example, two substantially identical devices indicating exposure to slightly different conditions but both exhibiting optimal assay performance would provide information relating an range of conditions for optimal assay performance. In the alternative, two substantially identical devices indicating exposure to slightly different conditions and different assay performance would provide information relating to the conditions that would affect assay performance.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains. All patents, patent applications, journal articles and other references cited herein are incorporated by reference in their entireties.

We claim:

1. A device comprising a substrate having a plurality of different molecular probes attached to a surface thereof and an integrated indicator that exhibits a response when exposed to a condition to which the substrate may be exposed,
wherein each different molecular probe is selected to interact with a corresponding target, and further wherein the indicator response is detectable after removing the indicator from the condition.

2. The device of claim 1, wherein the indicator response to the condition is detectable for at least 1 minute after removing the indicator from the condition.

3. The device of claim 2, wherein the indicator response to the condition is detectable for at least 1 hour after removing the substrate from the condition.

4. The device of claim 3, wherein the indicator response to the condition is substantially permanently detectable.

5. The device of claim 1, wherein the condition is an environmental condition that allows for target-probe interaction.

6. The device of claim 5, wherein the environmental condition is a predetermined temperature.

7. The device of claim 6, wherein the predetermined temperature is a maximum temperature.

8. The device of claim 7, wherein the maximum temperature is about 60° C. to about 90° C.

9. The device of claim 6, wherein the predetermined temperature is a minimum temperature.

10. The device of claim 9, wherein the minimum temperature is about 35° C. to about 45° C.

11. The device of claim 5, wherein the environmental condition is a predetermined water content.

12. The device of claim 5, wherein the environmental condition is a chemical concentration.

13. The device of claim 12, wherein the chemical concentration is a formamide concentration,

14. The device of claim 12, wherein the chemical concentration comprises a pH of about 5 to about 9.

15. The device of claim 12, wherein the chemical concentration is a salinity of about 0.01 molar to about 8 molar.

16. The device of claim 1, wherein the condition is the presence of a chemical moiety that affects the target-probe interaction.

17. The device of claim 16, wherein the chemical moiety hinders the target-probe interaction.

18. The device of claim 16, wherein the chemical moiety enhances the target-probe interaction.

19. The device of claim 1, wherein the indicator response is optically detectable.

20. The device of claim 19, wherein the indicator response is fluorescently detectable.

21. The device of claim 1, wherein the indicator response is magnetically detectable.

22. The device of claim 1, wherein the indicator response is electrically detectable.

23. The device of claim 1, wherein the indicator response is machine detectable.

24. The device of claim 1, wherein the response occurs after exposure of the indicator to the condition for at least a predetermined period.

25. The device of claim 24, wherein the predetermined period is about 1 minute to about 28 hours.

26. The device of claim 25, wherein the predetermined period is about 5 to about 10 hours.

27. The device of claim 26, wherein the predetermined period is about 6 to about 8 hours.

28. The device of claim 1, wherein the molecular probes are biomolecular.

29. The device of claim 28, wherein the molecular probes are nucleotidic.

30. The device of claim 28, wherein the molecular probes are peptidic.

31. The device of claim 28, wherein the molecular probes are oligomeric.

32. The device of claim 28, wherein the molecular probes are polymeric.

33. The device of claim 1, wherein the molecular probes are arranged in an array on the substrate surface.

34. The device of claim 33, wherein the array comprises at least about 10 probes per square centimeter of substrate surface.

35. The device of claim 34, wherein the array comprises at least about 50,000 probes per square centimeter of substrate surface.

36. The device of claim 35, wherein the array comprises at least about 200,000 probes per square centimeter of substrate surface.

37. The device of claim 36, wherein the array comprises at least about 1,000,000 probes per square centimeters of substrate surface.

38. The device of claim 1, wherein the substrate further contains machine-readable information.

39. The device of claim 38, wherein the substrate further comprises a medium on which information may be written.

40. The device of claim 39, wherein the medium is selected to contain electronic information.

41. The device of claim 39 wherein the medium is noncoplanar with respect to the surface on which the molecular probes are attached.

42. The device of claim 41, wherein the medium is writable from a surface that opposes the surface on which the molecular probes are attached.

43. The device of claim 1, wherein the substrate comprises a disk.

44. The device of claim 1, wherein the substrate comprises a tape.

45. The device of claim 1, wherein the substrate comprises a well plate.

46. The device of claim 1, wherein the substrate comprises a slide.

47. The device of claim 1, wherein the targets represent portions of a single molecule.

48. The device of claim 1, wherein the targets represent portions of single cell.

49. The device of claim 1, wherein the integrated indicator comprises nucleotidic material.

50. A device comprising a substrate having a plurality of molecular probes attached to a surface thereof and a plurality of different integrated indicators, each indicator selected to exhibit a response when exposed to one of a plurality of conditions to which the substrate may be exposed, wherein the molecular probes are selected to interact with corresponding targets, and further wherein the response is detectable after removing the indicator from the condition.

51. The device of claim 50, wherein the molecular probes are selected to interact with corresponding targets when exposed to at least one of the plurality of conditions.

52. The device of claim 51, wherein the molecular probes are selected to interact with corresponding targets when exposed to all of the conditions.

53. The device of claim 52, wherein the molecular probes are selected to interact with corresponding targets when exposed to all of the conditions simultaneously.

54. A device comprising a substrate having a plurality of nucleotidic molecular probes attached to a surface thereof and an integrated indicator that exhibits a response when exposed to a condition to which the substrate may be exposed, wherein the nucleotidic molecular probes are selected to interact with corresponding targets, and further wherein the response is detectable after removing the indicator from the condition.

55. The device of claim 54, wherein the condition represents a hybridization condition between the probes and targets.

56. A device comprising a substrate having a surface adapted for attachment to a plurality of molecular moieties and an integrated indicator that exhibits a response when exposed to a condition, wherein the response is detectable after removing the indicator from the condition.

57. The device of claim 56, wherein the condition is suitable for attaching the plurality of molecular moieties to the substrate surface.

58. The device of claim 56, wherein the condition is not suitable for attaching the plurality of molecular moieties to the substrate surface.

59. An apparatus for attaching molecular moieties to the substrate surface of the device of claim 56, comprising:
an indicator-response detector for detecting whether the indicator exhibits the response to the condition; and
a means for attaching a plurality of molecular moieties to the surface of the substrate.

60. The apparatus of claim 59, wherein the attaching means is activated if the indicator-response detector detects the response to the condition.

61. A method for attaching molecular moieties to a substrate surface, comprising attaching a plurality of molecular moieties to the substrate surface if the integrated indicator of the device of claim 56 exhibits a response to the condition.

62. A method for attaching molecular moieties to a substrate surface, comprising attaching a plurality of molecular moieties to the substrate surface if the integrated indicator of the device of claim 56 does not exhibit a response to the condition.

63. An apparatus for assaying a sample using the molecular probes attached to substrate surface of the device of claim 1, comprising:
an applicator for applying a sample to the molecular probes; and
an indicator-response detector for detecting whether the indicator of the device of claim 1 exhibits a response.

64. The apparatus of claim 63, further comprising a interaction detector for detecting probe-target interactions.

65. The apparatus of claim 64, wherein the interaction detector is an optical detector.

66. The apparatus of claim 65, wherein the interaction detector is a fluorescence detector.

67. The apparatus of claim 64, wherein the interaction detector is a magnetic detector.

68. The apparatus of claim 64, wherein the interaction detector is a electric detector.

69. The apparatus of claim 64, wherein the interaction detector is activated when the indicator-response detector detects a response by the indicator.

70. The apparatus of claim 64, wherein the interaction detector is deactivated when the indicator-response detector detects a response by the indicator.

71. The apparatus of claim 63, wherein the indicator-response also serves as an interaction detector for detecting probe-target interactions.

72. A method for assaying a sample, comprising the steps of:
(a) exposing the device of claim 1 to an assay condition by contacting the sample with the molecular probes attached to the substrate surface of the device;
(b) detecting whether the indicator exhibits the response to the condition; and
(c) detecting for probe-target interactions if the indicator exhibits the response to the condition.

73. The method of claim 72, wherein step (a) comprises placing the sample and the device in a controlled environment.

74. The method of claim 73, wherein step (a) comprises heating the device while the sample is in contact therewith.

75. The method of claim 73, wherein step (a) comprises preventing the sample from evaporating.

76. The method of claim 72, further comprising, after step (a) and before step (c), (a′) removing excess sample from the device.

77. The method of claim 72, wherein steps (b) and (c) are carried out using a single reader.

78. The method of claim 72, further comprising, after step (b), (b′) recording whether the response occurred as information contained in the device.

79. The method of claim 72, further comprising, after step (c), (c′) recording whether the probe-target interaction occurred as information contained in the device.

80. A method for assaying a sample, comprising the steps of:
(a) exposing the device of claim 1 to an assay condition by contacting the sample with the molecular probes attached to the substrate surface of the device;
(b) detecting for probe-target interactions if the indicator does not exhibit the response to the condition.
